(19) United States
(12) Patent Application Publication
(10) Pub. No.: US 2008/0254144 A1
(43) Pub. Date: Oct. 16, 2008

(54) Beverage health formulations

(76) Inventor: Shahram A. Banayan, La Jolla, CA (US)

Correspondence Address: MICHAEL A. ERVIN, 8202 TALBOT COVE, AUSTIN, TX, 78746, US

(21) Appl. No.: 11/787,346

(22) Filed: Apr. 16, 2007

(51) Int. Cl. A61K 36/00; A61K 33/06; A61K 36/45; A61K 36/82; A61K 33/30

(52) U.S. Cl. 424/641; 424/600; 424/682; 424/729; 424/732; 424/766; 424/777

(57) ABSTRACT

Dietary supplement formulations are described that provide benefits of soluble fiber accompanied by excellent taste with many different health benefit combinations.

### BACKGROUND OF INVENTION

The instant invention relates to vitamins, minerals, and dietary supplements; health formulations that can be added to water to create beverages that provide for high absorption of healthy ingredients that are delivered with excellent taste. One aspect of this is health beverages that have unique combination of soluble fibers with sweetener combinations that give outstanding flavor coupled with low glycemic index and provide healthy fiber action for the body.

Most health organizations and the federal government recognize the importance of dietary fiber. The Dietary Guidelines for Americans published jointly by the United States Departments of Agriculture and Health and Human Services recommend eating foods that have adequate amounts of fiber. The National Cancer Institute recommends 20 to 30 grams of fiber per day with an upper limit of 35 grams.

Glycemic index has increasingly become recognized as a key metric in keeping blood sugar levels under better control and thus reducing or eliminating the “insulin response” that occurs when blood sugar spikes after ingestion of food or drink.

Dietary fiber is plant material that is resistant to breakdown (digestion) by the human digestive system. In general, there are two major kinds of dietary fiber-insoluble (cellulose, hemicellulose, lignin) and soluble (gums, mucilages, pectins). Insoluble fiber promotes normal elimination by providing bulk for stool formation and hastening the passage of the stool through the colon. Soluble fibers also plays a healthy role in digestion and blood sugar control by slowing down the processing of sugars in digestion and therby reducing the insulin response. Studies also indicate that soluble fibers may play a role in reducing the level of serum cholesterol in the blood. This could also be related to the insulin response as their are indications that insulin has a role in producing the production of triglycerides and unhealthy serum cholesterols in the liver.

Numerous attempts have been made to provide soluble fibers in beverages that provide the needed level of dietary soluble fiber while also delivering acceptable taste. Taste in this case is more than just an acceptable level of flavorings. Many sweetening and flavoring systems based on low sugar content have disappointed due to a flat flavor that does not appeal to the human tongue and often leave an unpleasant aftertaste. The addition of soluble fibers makes that a more challenging proposition.

There is a need then for flavorful compositions of soluble fiber based beverages that deliver pleasing tastes that encourage the consumption of soluble fibers.

### BRIEF SUMMARY OF INVENTION

The unmet need then is for fiber and sweetening compositions that have an appealing flavor with little aftertaste coupled with low glycemic index blended with various dietary supplements to give a beverage that provides for high absorption of healthy ingredients

A further aspect of the invention is the addition of helpful vitamins, minerals, and other dietary supplements to a soluble fiber drink that further enhance the health aspects of such a beverage.

These goals can be realized by use of a soluble fiber health formulation, including at least the dietary supplement Lo Han Kuo fruit extract; combined with a sweetener; a soluble fiber material.

### DESCRIPTION OF DRAWINGS

No drawings.

### DETAILED DESCRIPTION OF THE INVENTION

It has been found that a particular combination of a sweetener package with some soluble fibers leads to an especially flavorful combination that addresses the needs defined above. A naturally sweet fruit available from China called Lo Han Kuo has been used to create a fruit extract that when combined with some low calorie sweeteners provide an especially good sweetener package for soluble fibers in water.

A preferred embodiment of the instant invention is the particular combination of the soluble fiber inulin, erythritol sweetener, and Lo Han Kuo fruit extract. When combined with other dietary supplements, natural fruit flavors, citric acid, and natural colors a very useful, healthy, and flavorful beverage combination is available that provides a low glycemic index drink for health conscious consumers.

It has also been found that in addition to inulin the soluble fibers polydextrose and fibersol are useful for working in conjunction with Lo Han Kuo fruit extract and erythritol.

Although erythritol is a preferred embodiment as a sweetener to work in conjunction with Lo Han Kuo fruit extracts other useful sweeteners have been found to work well. These include aspartame, saccharin, sucralose, neoetame, alitame, cyclamate, FOS, sorbitol, mannitol, xilitol, D-xylose, acesulfame, Isomalt, stevia, sucrose, brown sugar, evaporated cane juice, cane juice. Any and all fruit juices, any and all fruit extracts, corn syrup, fructose, high fructose corn syrup, honey, maple syrup.

The following combinations are all aspects of the instant invention. They are proposed in two different formats. The first format is as described with Lo Han Kuo fruit extract and a soluble fiber in combination with different packages that provide benefits such as electrolyte replacement, antioxidant characteristics, strengthening of the immune system, support of healthy skin, support of human endurance, low carbohydrates, and low calories. The same combinations are then proposed with low glycemic index sweeteners without the Lo Han Kuo fruit extract and soluble fiber.

In one embodiment, in addition to the fiber, sweetener, and Lo Han Kuo fruit extract a formulation of active mineral complexes can be added to water and natural or artificial flavors to produce a healthful low calorie mineral enhanced beverage that provides optimal electrolyte replacement. The combination of these with a useful soluble fiber and sweetener package delivers healthy minerals to the end consumer.

In another embodiment, in addition to the fiber, sweetener, and Lo Han Kuo fruit extract a formulation comprising vitamins A, C, Niacin, B3, B5, B6, B12, calcium, magnesium, zinc, selenium, manganese, potassium, chromium, alpha lipoic acid, citrus bioflavonoid, grape seed and grape skin extracts, kudzu extract can be added to water and natural or artificial flavors as a high potency, low calorie immune defense beverage that strengthens the human immune system.

In another embodiment, in addition to the fiber, sweetener, and Lo Han Kuo fruit extract a formulation comprising CoQ10, citric acid, and vitamin E can be added to water and natural or artificial flavors to provide a beverage that has superior antioxidant characteristics.

In another embodiment, in addition to the fiber, sweetener, and Lo Han Kuo fruit extract a formulation comprising Vitamin C, calcium, grape seed extract, grape skin extract, citric acid, alpha lipoic acid, DMAE, acetyl-L-carnitine can be added to water and natural or artificial flavors to provide a beverage that supports healthy skin tissue.

In another embodiment, in addition to the fiber, sweetener, and Lo Han Kuo fruit extract a formulation comprising green tea, grape seed extract, grape skin extract, citrus bioflavonoid, citric acid, kudzu extract can be added to water and natural or artificial flavors to provide a beverage that has superior antioxidant characteristics.

In another embodiment, in addition to fiber, sweetener and Lo Han Kuo extract, a formulation comprising of white tea, grape seed and grape skin extracts, citrus bioflavonoid, citric acid, and kudzu extract, can be added to water and natural or artificial flavors to provide a low calorie beverage that has superior antioxidant characteristics

In another embodiment, in addition to the fiber, sweetener, and Lo Han Kuo fruit extract a formulation comprising black tea, grape seed extract, grape skin extract, citrus bioflavonoid, citric acid, and kudzu extract can be added to water and natural or artificial flavors to provide a low calorie beverage that has superior antioxidant characteristics.

In another embodiment, in addition to the fiber, sweetener, and Lo Han Kuo fruit extract a formulation comprising vitamins C, B3, B5, B6, B12, zinc, chromium, taurine, citric acid, and ribose can be added to water and natural or artificial flavors to provide a low calorie beverage that supports human endurance.

In another embodiment, in addition to the fiber, sweetener, and Lo Han Kuo fruit extract a formulation comprising pomegranate juice or extract, blueberry juice or extract and acai juice or extract, citrus bioflavonoid, grape seed and grape skin extracts, citric acid, and kudzu extract can be added to water and natural or artificial flavors to provide a low calorie beverage that has superior antioxidant characteristics.

In another embodiment, in addition to fiber, sweetener and Lo Han Kuo fruit extract, a formulation comprising protein, vitamins A, C, B3, B5, B6, B12, niacin, calcium, magnesium, manganese, zinc, selenium, citric acid, and potassium can be added to water and natural or artificial flavors to provide a low calorie beverage for weight loss support.

In another embodiment, in addition to fiber, sweetener and Lo Han Kuo fruit extract, a formulation comprising carbohydrates can be added to water and natural or artificial flavors to provide a low calorie carbohydrate beverage.

In another embodiment, in addition to the fiber, sweetener, and Lo Han Kuo fruit extract a formulation comprising Vitamins B3, B5, B6, B12, caffeine, guarana, L-carnitine, citric acid, and ginseng can be added to water and natural or artificial flavors to provide a low calorie beverage that provides energy.

In another embodiment, in addition to a low glycemic index sweetener, a formulation of active mineral complexes and citric acid can be added to water and natural or artificial flavors to produce a healthful low calorie mineral enhanced beverage that provides optimal electrolyte replacement. The combination of these with a useful soluble fiber and sweetener package delivers healthy minerals to the end consumer.

In another embodiment, in addition to a low glycemic index sweetener, a formulation comprising vitamins A, C, Niacin, B3, B5, B6, B12, calcium, magnesium, zinc, selenium, manganese, potassium, chromium, alpha lipoic acid, citrus bioflavonoid, grape seed and grape skin extracts, citric acid, and kudzu extract can be added to water and natural or artificial flavors as a high potency, low calorie immune defense beverage that strengthens the human immune system.

In another embodiment, in addition to a low glycemic index sweetener, a formulation comprising CoQ10, citric acid, and vitamin E can be added to water and natural or artificial flavors to provide a beverage that has superior antioxidant characteristics.

In another embodiment, in addition to a low glycemic index sweetener, a formulation comprising vitamin C, calcium, grape seed extract, grape skin extract, alpha lipoic acid, DMAE, acetyl-L-carnitine and citric acid can be added to water and natural or artificial flavors to to provide a beverage that supports healthy skin tissue.

In another embodiment, in addition to a low glycemic index sweetener, a formulation comprising green tea, grape seed extract, grape skin extract, citrus bioflavonoid, kudzu extract can be added to water and natural or artificial flavors to provide a beverage that has superior antioxidant characteristics.

In another embodiment, in addition to a low glycemic index sweetener, a formulation comprising of white tea, grape seed and grape skin extracts, citrus bioflavonoid, citric acid, and kudzu extract, can be added to water and natural or artificial flavors to provide a low calorie beverage that has superior antioxidant characteristics.

In another embodiment, in addition to a low glycemic index sweetener, a formulation comprising black tea, grape seed extract, grape skin extract, citrus bioflavonoid, citric acid, and kudzu extract can be added to water to provide a low calorie beverage that has superior antioxidant characteristics.

In another embodiment, in addition to a low glycemic index sweetener, a formulation comprising vitamins C, B3, B5, B6, B12, zinc, chromium, taurine, and ribose can be added to water and natural or artificial flavors to provide a low calorie beverage that supports human endurance.

In another embodiment, in addition to a low glycemic index sweetener, a formulation comprising pomegranate juice or extract, blueberry juice or extract and acai juice or extract, citrus bioflavonoid, grape seed and grape skin extracts and kudzu extract can be added to water and natural or artificial flavors to provide a low calorie beverage that has superior antioxidant characteristics.

In another embodiment, in addition to a low glycemic index sweetener, a formulation comprising protein, vitamins A, C, B3, B5, B6, B12, niacin, calcium, magnesium, manganese, zinc, selenium, and potassium can be added to water and natural or artificial flavors to provide a low calorie beverage for weight loss support.

In another embodiment, in addition to a low glycemic index sweetener, a formulation comprising carbohydrates can be added to water and natural or artificial flavors to provide a low calorie carbohydrate beverage.

In another embodiment, in addition to a low glycemic index sweetener, a formulation comprising vitamins B3, B5, B6, B12, caffeine, guarana, L-carnitine and ginseng can be added to water and natural or artificial flavors to provide a low calorie beverage that provides energy.

The formulations mentioned above can all be converted into drinkable beverages by adding them to water. Each of these formulations can also be prepared and offered to consumers as a powdered material ready to be added to water by the consumer and all of those combinations are also aspects of the instant invention.

While the invention has been described above with references to specific embodiments, it is apparent that many changes, modifications and variations in the materials can be made without departing from the inventive concept disclosed herein. Accordingly, the spirit and broad scope of the appended claims is intended to embrace all such changes, modifications and variations that may occur to one of skill in the art upon a reading of the disclosure.

1. A soluble fiber health formulation, comprising:
a. Lo Han Kuo fruit extract;
b. a sweetener; and
c. a soluble fiber material.

2. The soluble fiber health formulation of claim 1 wherein said sweetener has a low glycemic index and is selected from the group consisting of: aspartame, saccharin, sucralose, neoetame, alitame, cyclamate, FOS, erythritol, sorbitol, mannitol, xilitol, D-xylose, acesulfame, isomalt, and stevia.

3. (canceled)

4. The soluble fiber health formulation of claim 1 wherein said soluble fiber material is selected from the group consisting of: fibersol, inulin, and polydextrose.

5. The soluble fiber health formulation of claim 1 wherein said soluble fiber material is inulin and said sweetener is erythritol.

6. The soluble health formulation of claim 1 further comprising an added component selected from the group consisting of vitamin A, vitamin B3, vitamin B5, vitamin B6, vitamin B12, vitamin C, vitamin D, and vitamin E.

7. The soluble fiber health formulation of claim 1 further comprising an added component selected from the group consisting of CoQ10, DMAE, actyl-L carnitine, kudzu extract, ribose, guarana, taurine, caffeine, ginseng, and alpha lipoic acid.

8. The soluble fiber health formulation of claim 1 further comprising an added component selected from the group consisting of calcium, magnesium, manganese, zinc, chromium, selenium, and potassium.

9. The soluble fiber health formulation of claim 1 further comprising an added component selected from the group consisting green tea, white tea, black tea, grape seed extract, grape skin extract, citric acid, and citrus bioflavonoids.

10. The soluble fiber health formulation of claim 1 further comprising an added component selected from the group consisting of pomegranate juice, pomegranate extract, blueberry juice, blueberry extract, acai juice and acai extract.

11. The soluble fiber health formulation of claim 1 further comprising an added component selected from the group consisting carbohydrates and proteins.

12. The soluble fiber health formulation of claim 1 further comprising adding water to create a drinkable beverage.
